(19) United States
(12) Patent Application Publication
(10) Pub. No.: US 2012/0322131 A1
(43) Pub. Date: Dec. 20, 2012

(54) METHOD AND DEVICE FOR ANAEROBIC FERMENTATION

(75) Inventor: Holger Schneider, Flensburg (DE)

(73) Assignee: CONVIOTEC GMBH, Flensburg (DE)

(21) Appl. No.: 13/581,945

(22) PCT Filed: Feb. 28, 2011
(86) PCT No.: PCT/EP2011/052889

§ 371 Date: Aug. 30, 2012

(30) Foreign Application Priority Data
Mar. 4, 2010 (DE) 10 2010 010 294.6

(51) Int. Cl. C12P 5/02; C12M 1/00

(52) U.S. Cl. 435/167; 435/289.1

(57) ABSTRACT

The invention relates to a process for generating biogas, electrical energy and heat starting from biological materials, more precisely a process for the anaerobic fermentation of a flowable substrate using a reactor including at least: an inlet (1), and outlet (3), a plurality of divider walls (6) which divide at least the internal reactor volume provided for the substrate into a plurality of compartments (7 (i)-7 (iv)) and divide each individual compartment (7 (i)-7(iv)) into at least two chambers (8 (i)-8 (iv); 9 (i)-9 (iv)) through which the substrate flows in opposite directions, wherein the process is characterized in that for increasing or reducing a ratio of a volume of the chambers (8 (i)-8 (iv)) through which substrate flows in one direction to a volume of the chamber (9 (i)-9 (iv)) through which substrate flows in the other direction at least some of the divider walls (6) are arranged moveable with respect of their spatial location and/or position and/or extension, wherein the movement and/or extension of the divider walls (6) is controlled as a function of the dry substance content of the flowable substrate. The invention also relates to a reactor that is used for the process according to the invention.

### CROSS REFERENCE TO RELATED APPLICATIONS

This application is the U.S. National Stage of International Application No. PCT/EP2011/052889 filed Feb. 28, 2011, which claims priority to German Appl. No. 10 2010 010 294.6 filed Mar. 4, 2010.

### BACKGROUND OF THE INVENTION

1. Field of the Invention

The invention relates to a process for generating biogas, electrical energy and heat from biological materials, more precisely a process for the anaerobic fermentation of a flowable substrate using a reactor including at least:
an inlet (1),
an outlet (3),
a plurality of divider walls (6) which divide at least an internal reactor volume provided for the substrate into a plurality of compartments (7 (i)-7 (iv)) and divide each individual compartment (7 (i)-7(iv)) respectively into at least two chambers (8 (i)-8 (iv); 9 (i)-9 (iv)) that are flowed through by the substrate in opposite directions,

The invention also relates to a reactor that is used for the process according to the invention.

2. Description of Related Art

The generation of biogas from biological base material without oxygen (anaerobic) can be divided into four essential steps:

in a first step, designated “hydrolysis”, the complex compounds of the substrate material (e.g. carbohydrates, proteins, fats) are broken down into simpler organic compounds (e.g. amino acids, sugar, fatty acids). The bacteria involved in the process release enzymes which biologically break down the material.

in a second step, the intermediary products formed are broken down further in the so-called “acid forming phase” (acidogenesis) through acid forming bacteria into lower fatty acids (e.g. acetic acid, propion acid and butyric acid) and carbon dioxide (CO2) and hydrogen. Besides that, small amounts of lactic acid and alcohol are formed.

in a third step, subsequently the base products are transformed in “acetic acid formation” (acidogenesis) through bacteria into precursor substances of the biogas (acetic acid, hydrogen and carbon dioxide).

in the last step of biogas production, “methanogenesis”, methane is formed through bacteria from the products of acidogenesis.

When the four breakdown steps are jointly performed in one fermenter, this is designated a one-stage arrangement. Since the bacteria in the particular stages, however, have different requirements for their habitats, a spatial separation of the breakdown steps can be advantageous.

From WO 2006/124781, a biogas arrangement for anaerobic fermentation is known in which a reactor that is being used is divided into various fixated chambers in which the reactions described in paragraph [0004] occur.

In US 2007/0256971 A1, a transportable biogas arrangement is claimed which includes several chambers that are respectively fixated with respect to their sizes but flexible and formed as bladders.

Both known biogas arrangements have the disadvantage that they have to be configured in a particular manner for the damage propensity of the methane bacteria due to the fixated configuration of the biogas arrangements. A background of this is that the methane bacteria have the most sensitive reaction as a response to interferences from the list of all bacteria involved in anaerobic fermentation and they also only multiply very slowly. Therefore the habitat conditions within all prior art reactors are adapted to the methane bacteria.

The spatial separation of the breakdown stages recited supra, however, has limits since for example the bacteria of acetogenesis can depend from cohabitating with the bacteria of methanogenesis. The reason for this dependency is that the bacteria of acetogenesis do not tolerate excessive hydrogen content.

A particular problem of the prior art transportable arrangements with small fermenter volumes is their particularly high sensitivity to interferences, changes of the substrate composition which are naturally provided for biological materials and irregularities in the substrate supply quickly lead to changes of the ph value and to a change of the microbial population and thus to an instability of the system.

These problems are presently countered by using non-transportable large scale biogas arrangements which have continuously mixed reactors (CSTR-continuous stirred tank reactor) with up to several thousand cubic meters of operating volumes and long dwelling times. Also these systems still have considerable susceptibility against changes of the substrate composition.

In a continuously mixed reactor (CSTR-continuously stirred tank reactor), ideally identical fermentation and flow conditions prevail at all locations in the reactor. Thus, interferences immediately affect the entire reactor content. This process risk is kept as low as possible through a very low space loading (low substance supply per unit time) of the reactor.

### SUMMARY OF THE INVENTION

Thus, it is an object of the invention to provide a device in which the biogas process remains stable when the substrate composition and the substrate supply changes, wherein the device facilitates high yield per volume and time, effectively operates with short dwelling times, wherein the arrangement is controllable also for small fermenter volumes and runs with process stability even in transportable containers with low maintenance and monitoring requirements.

The object is achieved according to the invention through a method for anaerobic fermentation of a flow capable substrate with defined dry substrate content using a reactor, at least including:

an inlet (1),

an outlet (3),

a plurality of divider walls (6) which divide at least the internal reactor volume provided for the substrate into a plurality of compartments (7 (i)-7 (iv)) and divide each individual compartment (7 (i)-7(iv)) into at least two chambers (8 (i)-8 (iv); 9 (i)-9 (iv)) that are flowed through by the substrate in opposite directions, wherein the process is characterized in that

for increasing or reducing a ratio of the volume of the chambers (8 (i)-8 (iv)) through which substrate flows in one direction to the volume of the chamber (9 (i)-9 (iv)) through which substrate flows in the other direction at least some of the separating walls (6) are arranged moveably with respect of their spatial location and/or position and/or extension,

wherein the movement and/or extension of the divider walls (6) is controlled as a function of the dry-matter content of the flowable substrate.

Preferably, the reactor used for the proposed method is a steel container with a cube shape or a cylindrical shape, wherein the latter can be circular or elliptical. At least for smaller embodiments like e.g. experimental reactors, acrylic glass, plastic or fiber reinforced plastic is used as a construction material. For large reactors, concrete, steel and steel reinforced concrete are suitable for the base and for the sidewalls, and steel and fiber reinforced plastic materials are suitable for the roof without being limited to the configuration and/or the recited materials according to the present invention. With respect to the inner volume of the reactor, sizes of 4 liters are common for experimental reactors and up to 200 m³ for large reactors.

In a preferred embodiment of the method according to the invention, the reactor used includes a plurality of compartments (7 (i)-7 (iv)) positioned adjacent to one another along a longitudinal axis of the reactor in a preferred embodiment. In many experiments preceding the invention, in particular reactors were used for the method according to the invention in which each particular compartment (7 (i)-7 (iv)) was respectively divided into two chambers (8 (i)-8 (iv); 9 (i)-9 (iv)) flowed through by the substrate in a counteracting manner, wherein the chambers (8 (i)-8 (iv)), per compartment, the respective chamber flowed through by the substrate first, are flowed through by the substrate in downward direction and the chambers (9 (i)-9 (iv)), per compartment respectively the chamber flowed through by the substrate last are flowed through by the substrate in upward direction. Reactors of this type are preferred for the invention. Thus, in a particularly preferred manner, as a function of the dry substance content of the flow capable substrate, at least a portion of the divider walls (6) is movable along the direction of the longitudinal axis of the reactor. The partition of the reactor into a plurality of compartments (7 (i)-7 (iv)) and the subdivision of each particular compartment (7 (i)-7 (iv)) respectively into at least two chambers (8 (i)-8 (iv); 9 (i)-9 (iv)) is thus performed with a plurality of divider walls (6) which are preferably vertically oriented.

The methods proposed herein, however, by no means require a configuration of the reactors to be used with compartments (7 (i)-7 (iv)) positioned adjacent to one another along the longitudinal axis of the reactor and with movable divider walls (6) oriented along the longitudinal axis of the reactor in a vertical direction. Compartments (7 (i)-7 (iv)) arranged above one another are also conceivable with preferably horizontally arranged divider walls (6).

With respect to the divider walls (6) besides moving them, also rotating, pivoting or flipping them, extracting them and inserting them and a spatial expansion and tapering is also conceivable as a function of the dry substance content of the substrate, wherein in particular rotating, pivoting or flipping the divider walls are particularly advantageous embodiments for moving the divider walls besides just moving them in a linear manner.

Surprisingly it has become apparent that a condition of auto-immobilization of the bacteria in the respective chambers (9 (i)-9 (iv)) flowed through in upward direction of the particular compartments (7 (i)-7 (iv)) can be achieved when the distance of the divider walls (6) is adapted according to the invention to the dry substance content of the substrate used. Thus, the dry substance content of the substrate is preferably metered in the inlet (1) to the reactor. By the same token, a determination of the dry substance content of the substrate in the outlet (3) is feasible but requires more detailed knowledge of the process properties of the reactor.

During the inventive adaptation of the distance of the divider wall (6) to the content of dry substance of the substrate used, there is a decoupling of the hydraulic dwelling time from the solid material dwelling time. Through the controlled modification of the chambers (8 (i)-8 (iv); 9 (i)-9 (iv)) of the compartments (7 (i)-7 (iv)), a completely new method for controlling the process of the biogas production is achieved.

Table 1 illustrates a particularly advantageous ratio of the respective volume of the chambers (8 (i)-8 (iv)) flowed through by the substrate in downward direction to the volume of the chambers (9 (i)-9 (iv)) flowed through by the substrate in upward direction as a function of the dry substance content of the substrate for particularly stable biogas manufacturing processes according to the instant invention.

TABLE 1

| | Ratio of the respective volumes of the chambers (8 | |
|---|---|---|
| Dry | (i)-8 (iv)) flowed through by the substrate in | |
| Substance | downward direction to the respective volumes of | |
| Content in | the chambers (9 (i)-9 (iv)) flowed through by the | c.f. |
| % by weight | substrate in upward direction | FIG. |
| 2% | 1:3 | 2a |
| 2-5% | 1:2 | 2b |
| 5-10% | 1:1 | 2c |
| 10-15% | 2:1 | 2d |
| 15-20% | 3:1 | 2e |

It can be derived from the table that in the proposed method, advantageously the ratio of the respective volume of the chambers (8 (i)-8 (iv)) flowed through by the substrate in downward direction relative to the volume of the chambers (9 (i)-9 (iv)) flowed through by the substrate in upward direction is adjusted so that it increases with increasing dry substance content of the substrate.

It is particularly preferable when the ratio of the respective volume of the chambers (8 (i)-8 (iv)) flowed through by the substrate in downward direction to the volume of the chambers (9 (i)-9 (iv)) flowed through by the substrate in upward direction for a dry substance content of less than 2% by weight is in a range of [1:3.5] to [1:greater 2.5] for the method according to the invention.

It is particularly preferable when the ratio of the respective volume of the chambers (8 (i)-8 (iv)) flowed through by the substrates in downward direction to the volume of the chambers (9 (i)-9 (iv)) flowed through by the substrate in upward direction for a dry substance content of 2% to 5% by weight is in a range of [1:2.5] to [1:greater 1.5] for the method according to the invention.

It is particularly preferable when the ratio of the respective volume of the chambers (8 (i)-8 (iv)) flowed through by the substrates in downward direction to the volume of the chambers (9 (i)-9 (iv)) flowed through by the substrate in upward direction for a dry substance content of 5% to 10% by weight is in a range of [1:1.5] to [smaller 1.5:1] for the method according to the invention.

It is particularly preferable when the ratio of the respective volume of the chambers (8 (i)-8 (iv)) flowed through by the substrates in downward direction to the volume of the chambers (9 (i)-9 (iv)) flowed through by the substrate in upward direction for a dry substance content of 10% to 15% by weight is in a range of [greater 1.5:1] to [2.5:1] for the method according to the invention.

It is particularly preferable when the ratio of the respective volume of the chambers (8 (i)-8 (iv)) flowed through by the substrates in downward direction to the volume of the chambers (9 (i)-9 (iv)) flowed through by the substrate in upward direction for a dry substance content of 15% to 20% by weight is in a range of [greater 2.5:1] to [3.5:1] for the method according to the invention.

The invention also relates to a reactor as it is being used for the method according to the invention in at least one preferred embodiment. Thus the reactor for anaerobic fermentation of a flow capable substrate with defined dry substrate content includes at least:

an inlet (1),

an outlet (3),

a plurality of divider walls (6) which divide at least the internal reactor volume provided for the substrate into a plurality of compartments (7 (i)-7 (iv)) and which divided each individual compartment (7 (i)-7(iv)) into at least two chambers (8 (i)-8 (iv); 9 (i)-9 (iv)) which are flowed through by the substrate in opposite directions, where the proposed reactor is characterized in that

for increasing or reducing a ratio of a volume of the chambers (8 (i)-8 (iv)) through which substrate flows in one direction to the volume of the chamber (9 (i)-9 (iv)) through which substrate flows in the other direction

the divider walls (6) are arranged moveably in respect of their spatial location and/or position and/or extension, and

wherein the reactor includes at least one control for controlling the movement of the divider walls (6) as a function of the dry-matter content of the flowable substrate.

In a preferred embodiment, the divider walls (6) extend over the entire width of the reactor.

The divider walls (6) are advantageously arranged so that forming seepages and/or plugs within the reactor is prevented and an optimum flow through the reactor is permanently provided.

The floors of the particular compartments (7 (i)-7 (iv)) can be configured differently. They can be circular for example, or they can be straight with or without inclination. They can also be configured with one or plural extraction points for the substrate introduced into the reactor so that it is possible to retrieve substrate from the reactor at various locations and to reintroduce the substrate at other locations (recycling). In order to retrieve the substrate, it can be advantageous to use a pump. In particular, a mono-pump which is connected through different valves with all intermediary outlet and inlet locations can be used for recycling.

The flow through the reactor is provided hydraulically through arranging the inlet (1) and the outlet (3) according to the principle of communicating pipes or it is supported by one or plural pumps.

Gas cavities (5) are provided above the particular compartments (7 (i)-7 (iv)) which are either connected with one another or hermetically separated, so that a gas retrieval can be performed completely in a gas flow or through a central gas outlet (2) or separately in plural gas flows through plural gas outlets (2), for example one gas outlet (2) per compartment (7 (i)-7 (iv)).

The reactor according to the invention can be sized so that it is integrateable into a container and therefore transportable.

Before being introduced into the reactor according to the invention, the materials to be fermented can be treated in a suitable manner so that the average particle size is ≦5 mm.

The reactor according to the invention and the method according to the invention facilitate anaerobic fermentation, preferably of materials with a dry mass content of 2 to 20% and a CSB of 3,000 to 500,000 mg/l.

The portion of the non-fermentable substances in the supplied substrate preferably does not exceed a portion of 20% by weight in the dry mass.

The definitions regarding percent by weight in the description and in the patent claims respectively relate to the “atro” weight, this means absolutely dry weight portions.

### BRIEF DESCRIPTION OF THE DRAWINGS

For a fuller understanding of the nature and object of the present invention, reference should be had to the following detailed description taken in connection with the accompanying drawings, in which:

FIG. 1 illustrates an optional embodiment of the proposed reactor for anaerobic fermentation of a flow capable substrate with defined dry substrate content; and

FIGS. 2a-2e illustrate the ratios of the respective volumes of the chambers flowed through by the substrate in downward direction to the respective volume of the chambers flowed through by the substrate in upward direction.

### DETAILED DESCRIPTION OF THE INVENTION

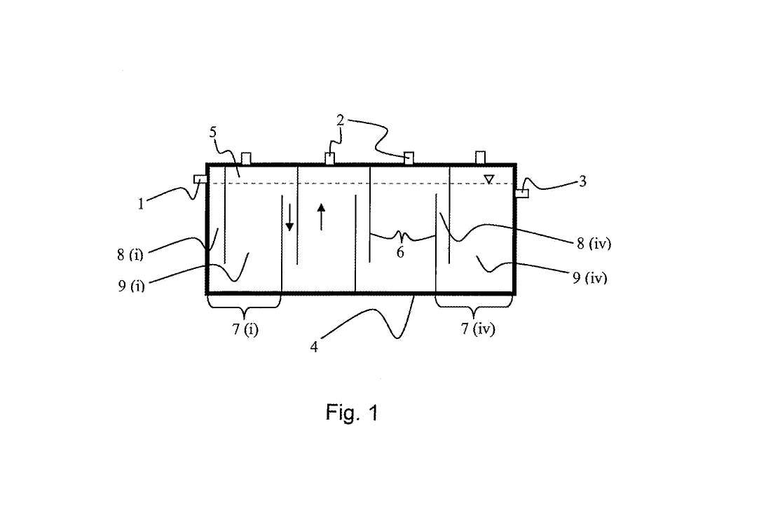

FIG. 1 illustrates an optional embodiment of the proposed reactor for anaerobic fermentation of a flow capable substrate with defined dry substrate content. Thus, the reactor is a cuboid container with horizontally arranged rectangular floor (4), vertical rectangular head- and sidewalls and a horizontally arranged roof. The reactor includes an inlet (1) in the upper portion of the first headwall and an outlet (3) in the upper portion of the opposite headwall, through which substrate can be introduced into the reactor and retrieved from the reactor. The interior of the reactor is divided into four compartments (7 (i)-7 (iv)) through three divider walls (6) that are not movable in the present embodiment and vertically extend from the base (4) into the inner cavity of the reactor. Through four movable divider walls (6) that are movable in this embodiment in a direction of the longitudinal axis of the reactor, each particular compartment (7 (i)-7 (iv)) is respectively divided into two chambers (8 (i)-8 (iv); 9 (i)-9 (iv)) that are being flowed through by the substrate in opposite directions. Thus, the chambers (8 (i)-8 (iv)) flowed through by the substrate first per compartment (7 (i)-7 (iv)) are flowed through by the substrate in downward direction and the chambers (9 (i)-9 (iv)) flowed through last by the substrate per compartment (7 (i)-7 (iv)) are flowed through by the substrate in upward direction. In the upper portion of each compartment (7 (i)-7 (iv)), a gas cavity (5) is configured with a particular gas outlet (2) through which the gases generated through anaerobic fermentation can be let out.

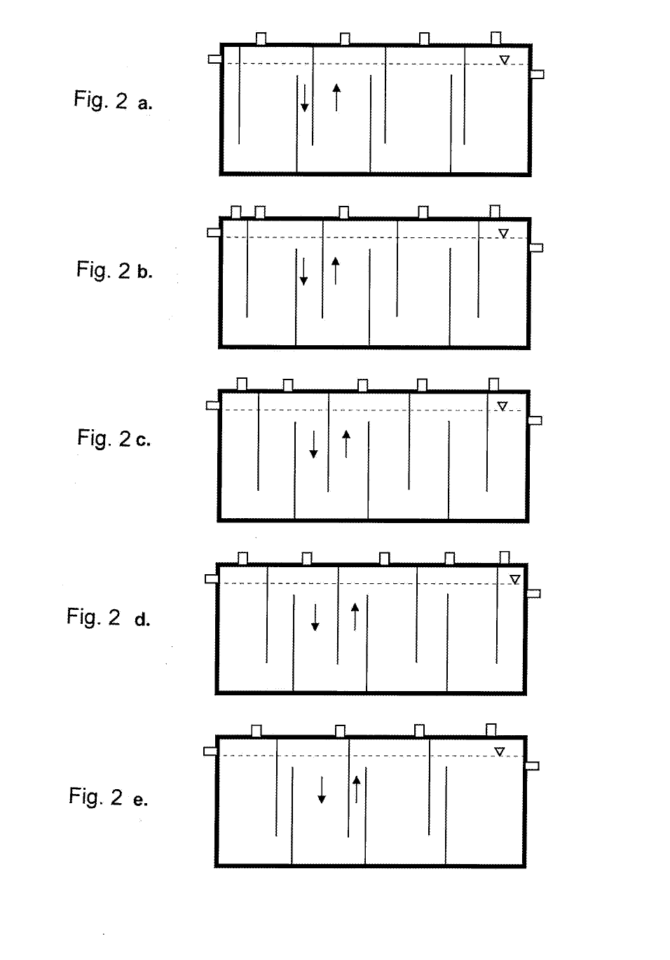

FIGS. 2a-2e illustrate for clarification purposes the ratios of the respective volumes of the chambers (8 (i)-8 (iv)) flowed through by the substrate in downward direction to the respective volume of the chambers (9 (i)-9 (iv)) flowed through by the substrate in upward direction, wherein the ratio can be influenced through moving the divider walls (6) for the cases illustrated in table 1.

For demonstrating the teachings according to the invention, the following tests were run which do not limit the general applicability of the teachings.

For comparison purposes, a conventional continuously stirred reactor (CSTR) with the following characteristics was used:
Operating volume: 4 liters
Head space: approximately 1 liter
Stirrer speed: 100 rpm
This lab reactor is comparable with a biogas fermenter with a central stirring arrangement and a height to diameter ratio (H:D) of 1:1.
   The maximum space loadings of the tested substrates for this lab reactor were the following:

Corn silage press juice: 7 kg oTS/m³ d

Food leftovers: 3 kg oTS/m³ d (foaming at higher loads)

Sugar beets: 7 kg oTS/m³ d

In all experiments with the conventional lab reactor, variations in the substrate supply (varying volumes, quality or so-called “shock loads”, sudden overloading with large substrate volumes) lead to a strong reduction of the biogas production until the biogas process comes to a complete standstill.

### Test Reactor According to the Invention

For deriving the parameters that are relevant for the invention, a small scale test reactor was used which in its basic configuration corresponds to the illustration in FIG. 1. This test reactor made from acrylic glass in the present embodiment has an operating volume of 4 liters with four compartments (7 (i)-7 (iv)) with one respective chamber flowed through in downward direction and one respective chamber flowed through in upward direction (8 (i)-8 (iv); 9 (i)-9 (iv)). The compartments (7 (i)-7 (iv)) respectively have a base surface of 0.002 m². The interior space of the reactor is 0.5 m tall and has an additional gas space (5) of 0.2 m. The volumes of the chambers (8 (i)-8 (iv); 9 (i)-9(iv)) depend from the respective ratio of the chambers (8 (i)-8 (iv)) flowed through in downward direction to the chambers (9 (i)-9 (iv)) flowed through in upward direction and are adapted according to the invention to the respective dry substance content of the substrate. The respective flow velocities in the particular chambers are listed. In a production scale bio reactor with several cubic meters, the flow velocities will be higher (up to 0.5 m per hour).

### Analysis Methods

The dry substance content of the substrates (TS) was gravimetrically determined by drying a sample at 105° C. over 24 hours (until the weight is constant) and is specified in percent solids. The organic dry substance (oTS) is the glowing loss of the dried sample which is generated when glowing the probe at 600° C. The oTS represents the percentage of organic substance with respect to the dry substance of the sample.

For determining the CBS values, cuvette kits (LCK 514) by Hach-Lange corporation were used.

The biogas yield was determined with a gas counter “milli-gas counter” by Ritter corporation.

### Processing of the Substrates

Due to the small flow through velocities and due to the low pump rates associated therewith of the hose pumps used it was necessary to pre-treat the substrates in a particular manner. Thus, the substrates were milled in a lab hammer mill with a screen diameter of 0.5 mm and homogenized.

### Results of the Test Fermentation

Particular test substrates were fermented in the test reactor at 37° C. with a vaccination culture added which came from gassed out slurry of an anaerobic stage of a municipal waste water treatment plant.

Substrates with a Dry Substance Content of 5 to 10%

(treatment with reactor according to the invention and application of the method according to the invention)
Test substrate: thermally pre-treated corn silage juice with 9% dry substance content
Dwelling Time: 8 days
Ratio of upward flowed through chamber to downward flowed through chamber: 1:1
Flow velocity in the downward flowed through chamber: 0.02 m/hr
Flow velocity in the upward flowed through chamber: 0.02 m/hr
CSB at reactor inlet: 120,000-140,000 mg/l
CSB at reactor outlet: 500-1,000 mg/l
Space loading: max. 11 kg oTS/m³ d
Biogas yield: 65 m³/ton of corn silage press juice

Substrates with a Dry Substance Content of 10 to 15%

(treatment with reactor according to the invention and application of the method according to the invention)
Test substrate: hygenized food left overs with 14.5% dry substance content
Dwelling Time: 10 days
Ratio of upward flowed through chamber to downward flowed through chamber: 2:1
Flow velocity in the downward flowed through chamber: 0.012 m/hr
Flow velocity in the upward flowed through chamber: 0.025 m/hr
CSB at reactor inlet: 200,000-230,000 mg/l
CSB at reactor outlet: 1,000-2,000 mg/l
Space loading: max. 15 kg oTS/m³ d
Biogas yield: 131 m³/ton of liquid pig manure

Substrates with a Dry Substance Content of 15 to 20%

(treatment with reactor according to the invention and application of the method according to the invention)
Test substrate: Cut up sugar beets with 19% dry substance content
Dwelling Time: 10 days
Ratio of upward flowed through chamber to downward flowed through chamber: 3:1
Flow velocity in the downward flowed through chamber: 0.01 m/hr
Flow velocity in the upward flowed through chamber: 0.033 m/hr
CSB at reactor inlet: 270,000-300,000 mg/l
CSB at reactor outlet: 1,000-2,000 mg/l
Space loading: max. 20 kg oTS/m³ d
Biogas yield: 153 m³/ton of sugar beets

In the method according to the invention, in all cases insensitivity relative to substrate variations becomes evident with respect to the volume and also with respect to the quality of the substrate.

The reactor according to the invention is robust against variations and interferences due to the compartmentalization. Thus, higher space loadings are possible in the reactor according to the invention. Different fermentation conditions like e.g. ph values are established in the particular compartments, wherein the fermentation conditions lead to a stabilization of the fermentation process. The biogas process includes a plurality of steps which build on each other but are performed under different conditions. The reactor according to the invention supports these particularities of the process. The higher space loadings, shorter dwelling times and the better fermentation conditions overall lead to higher yields per unit time (increases space—time yield) compared to conventional reactors as can be derived from the subsequent table 2.

TABLE 2
Increase of the space-time yield of the lab reactor according
to the invention a compared with a conventional lab reactor

| | Space-Time-Yield [m³ biogas | | |
|---|---|---|---|
| | per m³ reactor volume and day] | | Increase |
| | Lab reactor according | Conventional | of Space- |
| Test substrate | to the invention | Lab Reactor | Time-Yield |
| Liquid Pig Manure | 2 | 0.7 | 3x |
| Pre-treated corn | 9 | 3 | 3x |
| silage press juice | | | |
| Hygenizised food | 14 | 2 | 7x |
| leftovers | | | |
| Ground up sugar | 16 | 4 | 4x |
| beets | | | |

### REFERENCE NUMERALS AND DESIGNATIONS

1 inlet
2 gas outlet
3 outlet
4 base
5 gas cavity
6 divider walls
7 (i)-7 (iv) compartments (i) to (iv)
8 (i)-8 (iv) chambers in compartments (i) to (iv) flowed through in downward direction
9 (i)-9 (iv) chambers in compartments (i) to (iv) flowed through in upward direction

1. A method for anaerobic fermentation of a flowable substrate with a defined dry substance content using a reactor including at least:
an inlet (1),
an outlet (3),
a plurality of divider walls (6) which divide at least an internal reactor volume provided for the substrate into a plurality of compartments (7 (i)-7 (iv)) and which divide each individual compartment of the plurality of compartments (7 (i)-7(iv)) respectively into at least two sets of chambers (8 (i)-8 (iv); 9 (i)-9 (iv)) flowed through by the substrate in opposite directions,
wherein at least a portion of the divider walls (6) is arranged moveable with respect of their spatial location and/or position and/or extension for increasing or reducing a ratio of a volume of a first set of chambers (8 (i)-8 (iv)) of the at least two sets of chambers through which the substrate flows in one direction to a volume of a second set of chambers (9 (i)-9 (iv)) of the at least two sets of chambers through which the substrate flows in another direction,
wherein the movement and/or extension of the divider walls (6) is controlled as a function of a dry substance content of the flowable substrate.

2. The method according to claim 1, wherein the plurality of compartments (7 (i)-7 (iv)) are positioned adjacent to one another along a longitudinal axis of the reactor.

3. The method according to claim 2, wherein the first set of chambers (8 (i)-8 (iv)) are flowed through by the substrate in downward direction and the second set of chambers (9 (i)-9 (iv)) are flowed through by the substrate in upward direction.

4. The method according to claim 2, wherein at least a portion of the divider walls (6) is arranged movable relative to their orientation along a direction of the longitudinal axis of the reactor as a function of the dry substance content of the flowable substrate.

5. The method according to claim 3, wherein a ratio of the respective volume of the first set of chambers (8 (i)-8 (iv)) flowed through by the substrate in downward direction to the ratio of the second set of chambers (9 (i)-9 (iv)) flowed through by the substrate in upward direction increases with increasing dry substance content of the substrate.

6. The method according to claim 3, wherein a ratio of the respective volume of the first set of chambers (8 (i)-8 (iv)) flowed through by the substrate in downward direction to the ratio of the second set of chambers (9 (i)-9 (iv)) flowed through by the substrate in upward direction for a dry substance content of 2% by weight is in a range of [1:3.5] to [1:greater 2.5].

7. The method according to claim 3, wherein a ratio of the respective volume of the first set of chambers (8 (i)-8 (iv)) flowed through by the substrate in downward direction to the ratio of the second set of chambers (9 (i)-9 (iv)) flowed through by the substrate in upward direction for a dry substance content of 2% by weight to 5% by weight is in a range of [1:2.5] to [1:greater 1.5].

8. The method according to claim 3, wherein a ratio of the respective volume of the first set of chambers (8 (i)-8 (iv)) flowed through by the substrate in downward direction to the ratio of the second set of chambers (9 (i)-9 (iv)) flowed through by the substrate in upward direction for a dry substance content of 5% by weight to 10% by weight is in a range of [1:1.5] to [less than 1.5:1].

9. The method according to claim 3, wherein a ratio of the respective volume of the first set of chambers (8 (i)-8 (iv)) flowed through by the substrate in downward direction to the ratio of the second set of chambers (9 (i)-9 (iv)) flowed through by the substrate in upward direction for a dry substance content of 10% by weight to 15% by weight is in a range of [greater 1.5:1] to [2.5:1].

10. The method according to claim 3, wherein a ratio of the respective volume of the first set of chambers (8 (i)-8 (iv)) flowed through by the substrate in downward direction to the ratio of the second set of chambers (9 (i)-9 (iv)) flowed through by the substrate in upward direction for a dry substance content of 15% by weight to 20% by weight is in a range of [greater 2.5:1] to [3.5:1].

11. A reactor for anaerobic fermentation of a flowable substrate with a defined dry substance content, the reactor comprising:
an inlet (1),
an outlet (3),
a plurality of divider walls (6) which divide at least an internal reactor volume provided for the substrate into a plurality of compartments (7 (i)-7 (iv)) and which divide each individual compartment of the plurality of compartments (7 (i)-7(iv)) respectively into at least two sets of chambers (8 (i)-8 (iv); 9 (i)-9 (iv)) flowed through by the substrate in opposite directions,
wherein at least a portion of the divider walls (6) is arranged moveable with respect of their spatial location and/or position and/or extension for increasing or reducing a ratio of a volume of a first set of chambers (8 (i)-8 (iv)) of the at least two sets of chambers through which the substrate flows in one direction to a volume of a second set of chambers (9 (i)-9 (iv)) of the at least two sets of chambers through which the substrate flows in another direction,
wherein the reactor includes at least a control for moving the divider walls (6) as a function of a dry substance content of the flowable substrate.

12. The reactor according to claim 11, wherein the plurality of compartments (7 (i)-7 (iv)) are positioned adjacent to one another along a longitudinal axis of the reactor.

13. The reactor according to claim 12, wherein the first set of chambers (8 (i)-8 (iv)) are flowed through by the substrate in downward direction and the second set of chambers (9 (i)-9 (iv)) are flowed through by the substrate in upward direction.

14. The reactor according to claim 12, wherein at least a portion of the divider walls (6) is arranged movable relative to their orientation along a direction of the longitudinal axis of the reactor.

15. The reactor according to claim 11, wherein the divider walls (6) extend over an entire width of the reactor.
